(19) United States
(12) Patent Application Publication
(10) Pub. No.: US 2002/0102229 A1
(43) Pub. Date: Aug. 1, 2002

(54) Product for and method of controlling odor

(76) Inventor: Paul Wegner, San Carlos, CA (US)

Correspondence Address: BENASUTTI, P.A., 17294 BERMUDA VILLAGE DRIVE, BOCA RATON, FL, 33487, US

(21) Appl. No.: 10/058,548

(22) Filed: Jan. 28, 2002

Related U.S. Application Data
(60) Provisional application No. 60/265,147, filed on Jan. 29, 2001.

(51) Int. Cl. A61K 7/075

(52) U.S. Cl. 424/70.21

(57) ABSTRACT

A product and process for the immediate removal of odors from materials, and the prevention of the production of additional odors over extended periods of time. Oxides are combined with nitrates. The oxides get rid of the existing odors and the nitrates keep the system aerobic; which keeps the production of new odors from forming.

### CROSS-REFERENCE TO RELATED APPLICATIONS

This application is a Continuation-in-Part of my prior application, entitled ODOR CONTROL METHOD, Serial No. 60/265,147, filed Jan. 29, 2001; the disclosure of which is incorporated herein by reference as if fully set forth.

### BACKGROUND OF THE INVENTION

1. Technical Field

This invention relates to the control of odors, and more particularly, to the immediate odor removal and prevention of the production of additional odor by materials over extended periods of time.

2. Background Art

In the prior art, the collection of biological materials such as human and animal wastes and the processing of those materials in lagoons and otherwise, creates foul odors. The waste water generated from the cleaning animal containment areas, food processing plants, slaughter houses, portable toilets, and oil processing plants contain high levels of organic material, which may contain odoriferous chemicals or produce odors by the action of bacteria. Bacteria are usually the main cause of odor production. Much has been done in the prior art to treat those materials and to eliminate the odors these materials produce.

USFilter Distribution Group has a patent (RE36,651) that discloses the use of nitrates to remove foul odors. It states that aerobic bacteria must be present, nitrates must be present, and a certain amount of time is required before the odors can be removed. The body of the patent describes how aerobic bacteria use nitrates as an oxygen source to oxidize odoriferous compounds, such as mercaptans. The patent acknowledges that the biological oxidation of odiferous material is slow and requires a significant amount of time. It also acknowledges that the prior art teaches the use of nitrates to inhibit the production of odors from anaerobic bacteria.

The Proctor and Gamble patent (U.S. Pat. No. 6,287,550) teaches the inhibition of odors through the use of three measures:

1) Placing materials that inhibit the formation of odor via material that inhibit various bacteria, enzymes, or pH buffering agents.

2) Placing materials that absorb odors.

3) Placing materials that absorb liquids.

They also disclose the use of fresheners that must contain perfume in combination with materials that inhibit the formation of odors. The patent discloses the need for perfumes to mask odors; and only focuses on the inhibition of odor formation by the use of inhibitors of bacteria, enzymes, or buffering agents. It does not teach the use of nitrates, or materials such as metal oxides that favor the colonization of aerobic bacteria over anaerobic bacteria by providing an aerobic environment.

History has demonstrated that the complete inhibition or elimination of anaerobic bacteria over extended periods of time in an anaerobic environment is limited and unreliable at best, in open systems. Even the use of proven potent disinfectants such as chlorine, ultraviolet light, or ozone dissipate over time and the organic waste is soon colonized with anaerobic bacteria that are present in the air or in the waste materials themselves. Canned goods are an example of an anaerobic environment being maintained over extended periods of time, without the production of any foul odors. This is achieved with heat and no chemicals. In this case, the bacteria present are destroyed and all subsequent bacteria are excluded from the environment to prevent the formation of foul odors.

In an open environment, where bacteria are already present or subsequently inoculate the medium, the colonization of anaerobic bacteria is favored over aerobic bacteria as long as anaerobic conditions prevail. When anaerobic conditions prevail, aerobic bacteria are displaced by anaerobic bacteria. When aerobic conditions prevail, anaerobic bacteria are displaced by aerobic bacteria. The Procter and Gamble patent fails to address the need for creating an environment that favors aerobic bacteria, by avoiding the addition of organic compounds (which contribute to the oxygen demand) or the addition of agents such as nitrates to increase the supply of biologically available oxygen. Their primary focus is the inhibition of odor causing bacteria or enzymes.

The USFilter patent fails to provide for the instant removal of foul odors, while the Proctor and Gamble Patent fails to provide agents that provide a significant improvement in the supply of biologically available oxygen.

### DISCLOSURE OF THE INVENTION

### SUMMARY OF THE INVENTION

I have discovered a product for substantially eliminating existing foul odors (such as those associated with materials such as hydrogen sulfide) and substantially preventing the production of new foul odors in matter, which product comprises an agent which reacts with the existing foul odors and nitrate.

In this product, the agents which react with immediate foul odors are hydrogen peroxide and those agents represented by MO, where M=zinc, iron, calcium, magnesium, sodium, or potassium; and O=oxide, hydroxide, peroxide, carbonate, bicarbonate, percarbonate or silicate.

The nitrate sources are represented by MN, where N=nitrate and M=sodium, potassium, calcium, magnesium, ammonium, and aluminum.

The product is applied to a surface for odor control.

The product is applied to liquid absorbing material to produce a liquid absorbing material which has odor control properties.

Iron oxide or zinc oxide mixed with a paint binder, plaster or concrete produce an odor control surface. Nitrates are applied to the resulting surface.

A mixture of zinc oxide and calcium nitrate is used to control odor.

A mixture of iron oxide and calcium nitrate is used to control odor.

A mixture of hydrogen peroxide and calcium nitrate is used to control odor.

The product is added to an inorganic absorbent to produce a cat litter.

I have invented a product for substantially eliminating existing odors and substantially preventing the production of new odors in matter, comprising in combination oxide and nitrate. In its preferred form the oxide is selected from the group consisting of: hydrogen peroxide; iron oxide; and zinc oxide and the nitrate is calcium nitrate.

The oxides get rid of the existing odors and the nitrates keep the system aerobic; which keeps the production of new odors from forming.

I have discovered that oxides, which are not subject to decomposition, react with sulfur-based compounds, such as mercaptans, and immediately form reduced odoriferous materials. These are particularly effective on surface areas.

When these oxides, such as iron oxide and zinc oxide, are combined with nitrates in lagoons, the bacteria which feed on them prevent the system from going anaerobic.

In the prior art, it was known to use nitrates, but not oxides. I found that nitrates promote the colonization of aerobic bacteria and allow odor control over a long period of time.

Also I discovered that hydrogen peroxide gets rid of existing odors and, when combined with calcium nitrate, ensures that the system is aerobic.

When hydrogen peroxide is combined with nitrates in lagoons, existing odors are reduced and the aerobic bacteria that feed on the nitrates remain the dominant culture, as long as nitrates are present.

In the prior art, it was known to use nitrates, but not in combination with hydrogen peroxides or metal oxides. I have found that nitrates promote the colonization of aerobic bacteria which results in long term odor control by inhibiting the anaerobic bacterial production of odiferous chemicals. The hydrogen peroxide or metal oxides react with odiferous chemicals already present or introduced from time to time. This situation exists in the case of a cat litter box whereby odiferous compounds are introduced each time a cat urinates or defecates and the majority of odiferous compounds are produced by bacterial action over time.

### DESCRIPTION OF THE PREFERRED EMBODIMENTS

The preferred agents for the immediate removal of odiferous compounds are environmentally nontoxic, do not create toxic compounds, are noncorrosive, are nondiscoloring, do not produce foul odors, are odorless, do not cause allergic reactions, are inexpensive, do not add to the bacterial carbon food supply, have a pH between 4 and 8, do not damage materials that they come into contact with, and decompose into environmentally benign materials.

Hydrogen peroxide rapidly decomposes many odiferous compounds. Zinc oxide, iron oxide, calcium oxide, magnesium oxide and other metallic oxides react with many odiferous compounds to form less odiferous compounds. Hydrogen peroxide is the least toxic, followed by iron oxide, and zinc oxide. The metallic hydroxides are similarly effective. The metallic oxides, carbonates, and hydroxides are more stable than hydrogen peroxide. Hydrogen peroxide and zinc oxide are less discoloring than iron oxide. All have a pH between 3 and 9, are odorless, do not produce unpleasant odors, and contain no carbon. A pH approaching the acidic side of neutral (5-7) is preferred. Water soluble iron salts tend to be corrosive and staining. Metallic oxides such as lime are less preferred because they are corrosive and excessively alkaline. The high alkalinity can produce other undesirable odors such as ammonia.

The metallic hydroxides, peroxides, carbonates, percarbonates, bicarbonates, and silicates are similarly effective. The peroxides and percarbonates are preferred, by lack long term stability and tend to be more expensive. The balance of the metallic compounds are characterized by a metallic ion combined with a weakly acid ion such that sulfide compound can easily form.

The amount of the chemicals required is proportional to the amount of fresh foul odor present.

Perfumes and chlorine (which contain no organic carbon) containing compounds are unacceptable to many people, especially those who have allergies. Chlorine based compounds such as chlorites can produce carcinogenic materials, produce unpleasant odors, and are toxic to some plants. They can decompose into chlorides which are toxic to many plants. Sodium chlorate is used as a weed killer.

In addition to providing chemicals that remove fresh odors, chemicals that provide biologically available oxygen over extended periods of time are added to the mixture. The preferred oxygen containing chemicals are nitrate materials in the following order of preference: calcium nitrate, magnesium nitrate, potassium nitrate, ammonium nitrate, sodium nitrate, nitric acid, and cellulose nitrate. Other nitrate materials, such as aluminum nitrate are useful, but more expensive. Zinc nitrate can be used as a supplement. It supplies oxygen and inhibits the colonization of many pathogenic bacteria. However, it can be environmentally toxic if used in excessive amounts.

Calcium nitrate is the most preferred. It is very inexpensive and widely available as a plant fertilizer. It occurs naturally in large amounts near the root zones of plants such as corn, under certain conditions. Nitrates play a large role in the ecological life cycle. Some organisms convert nitrate into nitrogen gas. The calcium forms various nontoxic compounds such as calcium carbonate or can be assimilated by living things. Ammonium nitrate is less preferred because the ammonia can add to biological oxygen demand and contribute to the release of ammonia.

The amount of nitrates required is a function of the total amount organic materials present, biological oxygen demand, rate of oxygen consumption, and the length of time that aerobic conditions need to prevail. For example, when organic waste is applied thinly over a large area of nonflooded land, no nitrates are required, because the oxygen from the air is transferred at a sufficient rate for aerobic conditions to prevail. Surfaces that are contaminated with only a small amount organic matter may only require materials that remove existing foul odors. If the organic matter content of a moving river is low enough, the dissolved oxygen in the water may be sufficient. As the level of organic matter increases, the rate of oxygen transfer from the air can not keep up with the increased biological oxygen demand. The amount of nitrates required increases to makeup for the oxygen deficiency required for a given length of odor control time. The longer the odor control time, the greater the amount of nitrates required up to the maximum amount. The maximum amount of nitrate required is approximately equal to the amount required to convert all the organic matter into carbon dioxide, nitrogen, and sulfates in an oxygen free atmosphere. This is many times larger than the amount required to oxidize existing foul odors.

Once oxygen levels are insufficient to provide aerobic conditions, the risk of odor production increases. The amount of hydrogen sulfides or mercaptans present has very little to do with the practical estimate of nitrates required for the management of foul odors, except, when no organic matter is present. In the vast majority of organic waste odor control situations, the nitrate requirements are dictated by biological oxygen demand, rate of oxygen consumption, and length of odor control time required.

Enzymes, bacteria, cellulose, and other organic materials are less preferred because they can ultimately contribute to the biological oxygen demand.

The nitrate sources can be applied as liquid salt solution from the saturation point of the salt to very dilute solution or a powder. The solution can be added to septic tanks, applied to surfaces such as garbage cans, dumpsters, and cat boxes, or to porous materials such as cat litter, agricultural waste, or soil. A 50% to 0.01% solution is preferred. In some cases, I have found that increasing the nitrate ion to a concentration of 5 to 20 PPM is sufficient to keep a lagoon aerobic. Calcium nitrate is preferred because of its low toxicity and corrosion inhibitor qualities. Note that the calcium nitrate is hygroscopic.

When solutions containing calcium nitrate are sprayed onto surfaces such as dumpsters and garbage cans, the liquid sticks to the surface and remains wet. This property makes odor control on vertical surfaces much easier.

Materials that remove existing odors can be combined with the nitrate source or applied sequentially. These materials can be introduced in the form of granules, paint, powders, suspensions, plaster, concrete, or solution. Zinc oxide, zinc carbonate, or zinc nitrate are preferred because they are colorless and odorless. Zinc oxide is the most preferred because its pH is close to neutral and can be added to paint, plaster, and concrete. Iron oxide or iron carbonate can be used. It is less desirable because of its dark color which limits its use in paint. Hydrogen peroxide is favored where immediate odor removal is desired and long term stability is not critical.

Actual ranges of percentage of oxides and nitrate are as follows:

99% to 0.01% existing foul odor reducing agents. (In some cases the majority of foul odors are fresh; in other cases they are very little.) Nitrates 99.99% to 1%.

The above chemical mixtures can be applied dry or with water as a carrier.

Additional listings of workable oxides and nitrates are as follows:

Existing odor reducing agents are hydrogen peroxide and those agents represented by MO, where M=zinc, iron, calcium, magnesium, sodium, or potassium and O=oxide, hydroxide, peroxide, carbonate, bicarbonate, percarbonate, or silicate.

Agents to maintain aerobic conditions are represented by MN, where N=nitrate and M=sodium, potassium, calcium, magnesium, ammonium, and aluminum.

A description of the methods of using the products is as follows:

A garbage spray formula is sprayed on the can until a wet looking surface appears. It is sprayed in cat litter whenever odors start to increase. One pint of this material can be mixed with any nonclumping absorbent or cat litter material to form an absorbent material which reduces odors already present and provides long term odor control. In the case of clumping formula, an odor control agent would need to be mixed dry or before the clumping is introduced. Spray formula is applied at a rate of 70 gallons per million gallons of cattle manure lagoon capacity. It can be introduced as a spray or merely dumped into the lagoon. The lagoon may be checked once a week. Sufficient nitrate levels are indicated when the presence of green algae is seen on the surface.

The paint, plaster, and concrete formulas are applied in the same manner in which these materials are typically applied. The spray formulas can be applied to these surfaces after they have cured for added odor control.

Spray formula 1: 2 parts calcium nitrate, 1 part 35% hydrogen peroxide, 7 parts waters

Spray formula 2: 20 parts calcium nitrate, 2 parts zinc nitrate, 78 parts water

Spray formula 3: 10 parts calcium nitrate, 1 part zinc oxide, 90 parts water, plus a suspension agent.

Spray formula 4: 10 parts calcium nitrate, 1 part iron oxide, 90 parts water, plus a suspension agent.

Paint formula 1: polyurethane paint is 2 parts iron oxide to 1 part polyurethane paint.

Paint formula 2: Mixing one part polyurethane paint clear with one part zinc oxide weight/weight gives just the right level of mat finish.

Plaster formula 1: A 50/50-iron oxide/Portland cement mixture cures into a very hard material after the addition of water.

Plaster formula 2: A 10/90 zinc oxide-Plaster of Paris is sufficient.

Spray formula for applying to the paint or plaster surface: 1 part calcium nitrate; 10 parts water.

Situations.

1. In controlling odors in a liquid waste such as septic tanks, portable toilets, or organic waste lagoons, both agents should be water soluble; such as, a mixture of hydrogen peroxide and calcium nitrates.

2. In controlling odors on surfaces, any of the spray formulas are effective. Any of the paint or plaster formulas are effective. Their effectiveness can be enhanced with any of the spray formulas.

3. In controlling odors with a liquid absorbing material, such as cat litter, odor control properties can be incorporated into the absorbent material by mixing the odor control agent as a dry material (granular or powder) or in a liquid form.

Cat Box and Garbage Can Spray Formula:

Zinc oxide 10 grams, calcium nitrate 200 grams, water 200 grams

(Addition of suspension aid can be handy.)

Cat Litter Formula:

Inorganic moisture absorbent material such as “Absorb It” 20 pounds,

Zinc oxide 10 grams, calcium nitrate 200 grams, water 200 grams.

My new product and my new methods are as follows:

A product for substantially eliminating existing odors and substantially preventing the production of new odors in matter, comprising in combination oxide and nitrate.

The oxide can be from the group consisting of: calcium oxide; magnesium oxide; and sodium oxide.

The oxide may be zinc oxide.

The oxide may be iron oxide.

The oxide may be hydrogen peroxide.

The oxide may be selected from the group consisting of hydroxides, carbonate, bicarbonate, percarbonate and silicate and the nitrate may be selected from the group consisting of: sodium, potassium, calcium, magnesium, ammonium, and aluminum nitrate.

The amount of oxide to nitrate may be proportional to the amount of fresh existing odors to the amount of material producing new odors.

The proportion of the oxide and nitrate ingredients may be as follows: 0.1% to 50% calcium nitrate; 0.1% to 99.9% hydrogen peroxide.

The proportion of the oxide and nitrate ingredients may be as follows: 0.1% to 99.9% calcium nitrate; 0.1% to 99.9% zinc oxide.

The proportion of the oxide and nitrate ingredients may be as follows: 0.1% to 99.9% calcium nitrate; 0.1% to 99.9% zinc oxide, and the product may further comprise a suspension agent.

The proportion of the oxide and nitrate ingredients may be as follows: 0.1% to 99.9% calcium nitrate; 0.1% to 99.9% iron oxide.

The product may be a polyurethane paint and comprise at least 0.1% to 70% iron oxide; and polyurethane paint.

The product may be a polyurethane paint comprising at least 0.1% to 70% zinc oxide; and polyurethane paint.

The product may be Portland cement comprising at least 0.1% to 50% iron oxide; and Portland cement.

The product may be plaster and comprise at least 0.1% to 70% iron oxide; and plaster of Paris.

The amount of required oxide should be proportional to the amount of existing odors the product is exposed to.

The product may comprise in proportion to 0.1% to 99.9% zinc oxide, 0.1% to 99.9% calcium nitrate; and further comprise a liquid absorbent material.

The product may comprise in proportion 0.1% to 99.9% iron oxide; 0.1% to 99.9% calcium nitrate; and further comprise an inorganic moisture absorbent material.

The amount of nitrate may be proportional to the amount required to maintain aerobic conditions over the required time period.

If the product is used in a lagoon, the nitrate ion should be 5 to 200 parts per million.

A method of substantially eliminating existing odors and substantially preventing the production of new odors in matter, comprising applying a product to said matter, which product comprises a combination of oxide and nitrate. In this method, the product may be those set forth above.

The method may comprise products which additionally comprise water and are applied as a spray.

Also the product may additionally comprise inorganic moisture absorbent material.

The method may comprise a product wherein the nitrate is in a solution not exceeding 50% nitrate.

What is claimed is:

1. A product for substantially eliminating existing odors and substantially preventing the production of new odors in matter, comprising in combination oxide and nitrate.

2. The product of claim 1 wherein the oxide is selected from the group consisting of: calcium oxide; magnesium oxide; and sodium oxide.

3. The product of claim 1 wherein the oxide is zinc oxide.

4. The product of claim 1 wherein the oxide is iron oxide.

5. The product of claim 1 wherein the oxide is hydrogen peroxide.

6. The product of claim 1 wherein the oxide is selected from the group consisting of hydroxides, carbonate, bicarbonate, percarbonate and silicate.

7. The product of claim 2 wherein the nitrate is selected from the group consisting of: sodium, potassium, calcium, magnesium, ammonium, and aluminum nitrate.

8. The product of claim 6 wherein the nitrate is selected from the group consisting of: sodium, potassium, calcium, magnesium, ammonium, and aluminum nitrate.

9. The product of claim 1 wherein the amount of oxide to nitrate is proportional to the amount of fresh existing odors to the amount of material producing new odors.

10. The product of claim 1 wherein the proportion of the oxide and nitrate ingredients is as follows: 0.1% to 50% calcium nitrate; 0.1% to 99.9% hydrogen peroxide.

11. The product of claim 1 wherein the proportion of the oxide and nitrate ingredients is as follows: 0.1% to 99.9% calcium nitrate; 0.1% to 99.9% zinc oxide.

12. The product of claim 1 wherein the proportion of the oxide and nitrate ingredients is as follows: 0.1% to 99.9% calcium nitrate; 0.1% to 99.9% zinc oxide, and further comprises a suspension agent.

13. The product of claim 1 wherein the proportion of the oxide and nitrate ingredients is as follows: 0.1% to 99.9% calcium nitrate; 0.1% to 99.9% iron oxide.

14. The product of claim 1 in which the product is a polyurethane paint and comprises at least 0.1% to 70% iron oxide; and polyurethane paint.

15. The product of claim 1 in which the product is a polyurethane paint comprising at least 0.1% to 70% zinc oxide; and polyurethane paint.

16. The product of claim 1 in which the product is Portland cement comprising at least 0.1% to 50% iron oxide; and Portland cement.

17. The product of claim 1 wherein the product is plaster and comprises at least 0.1% to 70% iron oxide; and plaster of Paris.

18. The product of claim 1 wherein the amount of required oxide is proportional to the amount of existing odors the product is exposed to.

19. The product of claim 1 wherein the product comprises in proportion to 0.1% to 99.9% zinc oxide, 0.1% to 99.9% calcium nitrate; and further comprises a liquid absorbent material.

20. The product of claim 1 wherein the product comprises in proportion 0.1% to 99.9% iron oxide; 0.1% to 99.9% calcium nitrate; and further comprises an inorganic moisture absorbent material.

21. The product of claim 1 wherein the amount of nitrate is proportional to the amount required to maintain aerobic conditions over the required time period.

22. The product of claim 1 for use in a lagoon wherein the nitrate ion is 5 to 200 parts per million.

23. A method of substantially eliminating existing odors and substantially preventing the production of new odors in matter, comprising applying a product to said matter, which product comprises a combination of oxide and nitrate.

24. The method of claim 23 wherein the oxide is selected from the group consisting of: calcium oxide; magnesium oxide; and sodium oxide.

25. The method of claim 23 wherein the oxide is zinc oxide.

26. The method of claim 23 wherein the oxide is iron oxide.

27. The method of claim 23 wherein the oxide is hydrogen peroxide.

28. The method of claim 23 wherein the oxide is selected from the group consisting of hydroxides, carbonate, bicarbonate, percarbonate and silicate.

29. The method of claim 24 wherein the nitrate is selected from the group consisting of: sodium, potassium, calcium, magnesium, ammonium, and aluminum nitrate.

30. The method of claim 28 wherein the nitrate is selected from the group consisting of: sodium, potassium, calcium, magnesium, ammonium, and aluminum nitrate.

31. The method of claim 23 wherein the amount of oxide to nitrate is proportional to the amount of fresh existing odors to the amount of material producing new odors.

32. The method of claim 23 wherein the proportion of the ingredients is as follows: 0.1% to 50% calcium nitrate; 0.1% to 99.9% hydrogen peroxide, and the product is applied as a spray.

33. The method of claim 23 wherein the proportion of the ingredients is as follows: 0.1% to 99.9% calcium nitrate; 0.1% to 99.9% zinc oxide, and the product is applied as a spray.

34. The method of claim 23 wherein the proportion of the ingredients is as follows: 0.1% to 99.9% calcium nitrate; 0.1% to 99.9% zinc oxide, and further comprises a suspension agent, and the product is applied as a spray.

35. The method of claim 23 wherein the proportion of the ingredients is as follows: 0.1% to 99.9% calcium nitrate; 0.1% to 99.9% iron oxide, and further comprises a suspension agent.

36. The method of claim 23 in which the product is a polyurethane paint and comprises at least 0.1% to 70% iron oxide; and polyurethane paint.

37. The method of claim 23 in which the product is a polyurethane paint comprising at least 0.1% to 70% zinc oxide; and polyurethane paint.

38. The method of claim 23 in which the product is Portland cement comprising at least 0.1% to 50% iron oxide; and Portland cement.

39. The method of claim 23 wherein the product is at least comprised of plaster of Paris and 0.1% to 70% zinc oxide.

40. The method of claim 23 wherein the product is at least 1 part calcium nitrate and additionally comprises water and the product is applied as a spray.

41. The method of claim 23 wherein the product comprises in proportion to grams, 10 grams zinc oxide, 200 grams of calcium nitrate; and additionally, comprises water.

42. The method of claim 23 wherein the product comprises in proportion to grams, 10 grams zinc oxide, 200 grams of calcium nitrate; and additionally water, plus an additional inorganic moisture absorbent material.

43. The method of claim 23 wherein the nitrate is in a solution not exceeding 50% nitrate.

44. The method of claim 23 for use in a lagoon wherein the nitrate ion is 5 to 200 parts per million.
